(19) United States
(12) Patent Application Publication
(10) Pub. No.: US 2024/0348455 A1
(43) Pub. Date: Oct. 17, 2024

(54) BLOCKCHAIN-BASED SUPERVISION METHOD FOR ELECTRONIC IMMUNITY PASSPORT

(71) Applicant: Nanjing University of Posts and Telecommunications, Jiangsu (CN)

(72) Inventors: Haiping HUANG, Jiangsu (CN); Haoyu JI, Jiangsu (CN); Fu XIAO, Jiangsu (CN); Ruchuan WANG, Jiangsu (CN); Wei ZHANG, Jiangsu (CN); Jiankuo DONG, Jiangsu (CN); Long CHEN, Jiangsu (CN); Qi LI, Jiangsu (CN)

(73) Assignee: Nanjing University of Posts and Telecommunications, Jiangsu (CN)

(21) Appl. No.: 18/022,519

(22) PCT Filed: Sep. 1, 2022
(86) PCT No.: PCT/CN2022/116446

§ 371 Date: Jul. 4, 2024

(30) Foreign Application Priority Data
Oct. 21, 2021 (CN) 202111230252.X

(51) Int. Cl. H04L 9/32; H04L 9/00; H04L 9/08

(52) U.S. Cl. CPC H04L 9/3263; H04L 9/0861; H04L 9/3236; H04L 9/50

(57) ABSTRACT

The invention provides a blockchain-based supervision method for an electronic immunity passport. A certificate authority generates and correspondingly distributes public-private key pairs. A hospital generates an immunity passport for a vaccine and assigns a passport number; encrypts the immunity passport and the passport number; and generates and uploads a transaction to a national alliance chain node (NACN). The NACN generates and uploads a second transaction to a world alliance chain (WAC). When the vaccine is requested to show the immunity passport, the vaccinee provides the passport number, generates and uploads a trapdoor to the WAC. The vaccine decrypts received ciphertext from the WAC to obtain plaintext data. An alliance chain node performs periodic maintenance by a smart contract. The invention can ensure data openness, traceability, and immutability, achieve effective supervision for the immunity passport data, provide reliable immunity passport data, and achieve search and regular maintenance for the immunity passport.

### CROSS-REFERENCE TO RELATED APPLICATION

This application is a 371 of international application of PCT application serial no. PCT/CN2022/116446, filed on Sep. 1, 2022, which claims the priority benefit of China application serial no. 202111230252.X, filed on Oct. 21, 2021. The entirety of each of the above-mentioned patent applications is hereby incorporated by reference herein and made a part of this specification.

### TECHNICAL FIELD

The present invention provides a blockchain-based supervision method for an electronic immunity passport, which relates to the technical fields such as blockchain, consensus algorithms, and data security protection.

### BACKGROUND

After the global outbreak of COVID-19 pandemic, exit-entry control policies have been tightened around the world, restricting cross-border flow. The spread of the epidemic has brought a great impact on the development of international communication and international trade and economic activities. In order to restore and develop the economy, relieve the pressure on people's livelihood, and restore normal production and international communication, countries and regions successively propose issuing immunity passports.

However, the immunity passports have some problems. Due to the difference between different vaccine types, the different vaccination methods in different countries, and the time interval between multiple doses of the vaccine, disagreement has arisen among countries over the reliability of the immunity passports, increasing the difficulty of unified management.

On the other hand, it is also necessary to determine the minimum length of immunity after vaccination and to monitor its duration, so as to know whether and when it is required to reassess the immune status of a passport holder and update his/her passport. Moreover, the immunity passport has problems of forgery and privacy leakage of personal information data.

These are the issues that should be considered and addressed during the design and use of the immunity passport.

### SUMMARY

The present invention provides a blockchain-based supervision method for an electronic immunity passport, which solves the problems that the immunity passport data is not shared and its reliability needs to be ensured by means of blockchain and searchable encryption technologies, and achieves secure mutual recognition and communication of immunity passports between countries, so as to promote international exchange and gradual recovery of international trade and economic activities.

A blockchain-based supervision method for an electronic immunity passport is provided, which includes the following steps:
S1. initializing, by a certificate authority (CA), common parameters CP according to safety parameters; and generating public-private key pairs corresponding to various members, including a vaccinee i, a hospital h, a national alliance chain node c and a world alliance chain node w, in a blockchain system, and correspondingly distributing these key pairs;
S2. after the vaccinee i is vaccinated in the hospital h, generating, by the hospital h, an immunity passport m for the vaccinee i and assigning a passport number PUIDᵢ;
S3. encrypting, by the hospital h, the immunity passport m and the passport number PUIDᵢ, generating a hash value H₁(C₁) of ciphertext C₁ of the immunity passport m, and uploading the ciphertext C₁ of the immunity passport m to a hospital server for storage;
S4. generating, by the hospital h, a transaction T_{C} by means of the ciphertext hash value H₁(C₁), a keyword index I_{K}, and the date of expiry T of the immunity passport; and uploading the transaction T_{C} to the national alliance chain node;
S5. establishing, by the national alliance chain node, a safety index I_{S} by means of searchable encryption technology, generating a transaction T_{W} according to the safety index I_{S} and the date of expiry T, and uploading the transaction T_{W} to the world alliance chain;
S6. when the vaccinee i is requested to show the immunity passport m, generating, by the vaccinee i, a trapdoor T_{Q} by means of searchable encryption technology and according to the passport number PUIDᵢ, and then uploading the trapdoor T_{Q} to the world alliance chain as a transaction;
S7. after receiving the trapdoor T_{Q}, searching, by the world alliance chain, for the ciphertext C₁ of the corresponding immunity passport m by means of a smart contract, and sending the found ciphertext C₁ of the immunity passport m to the vaccinee i;
S8. decrypting, by the vaccinee i, the received ciphertext C₁ of the immunity passport to obtain plaintext data m of the immunity passport;
S9. performing, by an alliance chain node, including a national alliance chain node and a world alliance chain node, periodic maintenance by means of a smart contract; checking whether the immunity passport m expires; and updating the expired immunity passport m.

Further, step S1 is specifically as follows:
S11. selecting, by the CA, two multiplicative cyclic groups G₁ and G₂ of which the order is a prime p according to the input safety parameter λ, a generating element being g∈G₁; defining bilinear mapping e: G₁×G₁→G₂; setting three collision-proof hash functions: H₁: {0,1}*→Z*ₚ, H₂: {0,1}*→G₁, H₃: G₂→{0,1}*; and issuing the common parameters CP={p, g, e, H₁, H₂, H₃};
S12. randomly selecting, by the CA, xᵢ∈Z*ₚ as a private key skᵢ of the vaccinee i, and calculating the corresponding public key pkᵢ=g^{xi}; randomly selecting xₕ∈Z*ₚ as a private key skₕ of the hospital h, and calculating the corresponding key pk_{c}=g^{xc}; randomly selecting x_{c}∈Z*ₚ as a private key sk_{c} of the national alliance chain node c and calculating the corresponding key pk_{c}=g^{x}^{_{c}}; and randomly selecting x_{w} ∈Z*ₚ as a private key sk_{w} of the world alliance chain node w and calculating the corresponding key pk_{w}=g^{x}^{_{w}}; and
S13. correspondingly assigning, by the CA through a secure channel, the public-private key pairs obtained in step S12 to the members, including the vaccinee i, the hospital h, the national alliance chain node c and the world alliance chain node w, in the blockchain system.

Further, in step S2, the immunity passport m includes personal information and vaccination information of a user, the vaccination information including vaccination time, vaccination location, and vaccine information.

Further, in step S3, the hospital h encrypting the immunity passport m and the passport number PUIDᵢ is specifically as follows: randomly selecting, by the hospital h, a secrete value θ∈Z*ₚ, where Z*ₚ is the remainder class of the integer module p, that is, Z*ₚ={1,2, . . . , p-1}; calculating A=pkᵢ^{θ}, k=e(g^{θ},H₂(PUIDᵢ)), B=k×m, and F=H₃(k), where pkᵢ is the public key of the vaccinee i, e is the bilinear mapping defined in step S11, g is the generating element of G₁, G₁ is the multiplicative cyclic group of which the order is a prime p, H₂ and H₃ are two collision-proof hash functions, and m is the plaintext data of the immunity passport; and recording C₁=(A,B) as the ciphertext of the immunity passport m and C₂=(A,F) as the ciphertext of the immunity passport number PUIDᵢ.

Further, in step S4, the hospital h generating the transaction T_{C} by means of the hash value of the ciphertext H₁(C₁), the keyword index I_{K}, and the date of expiry T of the immunity passport and uploading the transaction T_{C} to the national alliance chain node is specifically as follows:
S41. establishing, by the hospital h, a keyword index I_{K}= [symbol] C [symbol] by means of searchable encryption technology and according to the ciphertext C₂ of the immunity passport number PUIDᵢ;
S42. generating, by the hospital h, a transaction T_{C} by means of the hash value H₁(C₁) of the ciphertext of the immunity passport m, the date of expiry T of the immunity passport, and the keyword index I_{K}; and
S43. packing, by the hospital h, the transaction T_{C} and uploading the same to the national alliance chain node; and running, by the national alliance chain node, a PBFT consensus mechanism to authenticate the transaction T_{C}, and generating a new block Block_{C} after successful authentication.

Further, in step S5, the national alliance chain node establishing a safety index I_{S} by means of searchable encryption technology, generating a transaction T_{W} according to the safety index I_{S} and the date of expiry T, and uploading the transaction T_{W} to the world alliance chain is specifically as follows:
S51. in each national alliance chain, extracting, by the national alliance chain node, a keyword index I_{K}, the data of expiry T, and a block identifier BID of the national alliance chain where the node is located from each transaction in the block Block_{C} generated in step S43;
S52. establishing a safety index I_{S}= [symbol] BID,I_{K} [symbol] by means of the keyword index I_{K} and the national alliance chain block identifier BID;
S53. generating, by the national alliance chain node, a transaction T_{W} by means of the safety index I_{S} and the date of expiry T; and
S54. packing and uploading, by the national alliance chain node, the transaction T_{W} to the world alliance chain; and running, by the world alliance chain node, a PBFT consensus mechanism to authenticate the transaction, and generating a new block Block_{W} after successful authentication.

Further, in step S6, the vaccinee i generating the trapdoor T_{Q} by means of searchable encryption technology and according to the passport number PUIDᵢ when the vaccinee i is requested to show the immunity passport m, and then uploading the trapdoor T_{Q} to the world alliance chain as a transaction is specifically as follows:
S61. when the vaccinee i is requested to show the immunity passport m, randomly selecting, by the vaccinee i, a secrete value d∈Z*ₚ, where Z*p={1,2, . . . , p-1}; and calculating τ₁=

$${{H}_{2} ( {PUID}_{i} )}^{\frac{1}{{sk}_{i}}} {pk}_{w}^{d}$$

and τ₂=g^{d} by means of his/her own passport number PUIDᵢ, where skᵢ is a private key of the vaccinee i, pk_{w} is a public key of the world alliance chain node w, H₂ is a harsh function defined in step S11, and g is a generating element of G₁; and
S62. generating a trapdoor T_{Q}= [symbol] τ₁, τ₂ [symbol] , where τ₁ and τ₂ are parameters calculated in step S61; and then uploading the trapdoor T_{Q} to the world alliance chain as a transaction.

Further, in step S7, the world alliance chain searching for the ciphertext C₁ of the corresponding immunity passport m by means of a smart contract after receiving the trapdoor T_{Q}, and sending the found ciphertext C₁ of the immunity passport m to the vaccinee i is specifically as follows:
S71. after receiving the trapdoor T_{Q}= [symbol] τ₁, τ₂ [symbol] , calculating, by the world alliance chain node,

$$\frac{{τ}_{1}}{{{τ}_{2}}^{{sk}_{w}}} ,$$

where sk_{w} is a private key of the world alliance chain node w, and

$${τ}_{1} = {{H}_{2} ( {PUID}_{i} )}^{\frac{1}{{sk}_{i}}} {pk}_{w}^{d}$$

and τ₂=g^{d} are parameters calculated in step S61;
S72. extracting, by the world alliance chain node, the safety index I_{S}= [symbol] BID, I_{K} [symbol] from the transaction T_{W} in the block Block_{W} generated in step S54, to authenticate whether the equation

$$F = {H}_{3} ( e ( A , \frac{{τ}_{1}}{{{τ}_{2}}^{{sk}_{w}}} ) )$$

holds true, where BID is the national alliance chain block identifier, I_{K}= [symbol] C₂ [symbol] is the keyword index, C₂=(A,F) is the ciphertext of the immunity passport number PUIDᵢ, A=pkᵢ^{θ} and F=H₃(k) are parameters calculated in step S3, k=e(g^{θ}, H₂(PUIDᵢ)), sk_{w} is the private key of the world alliance chain node w, τ₁ and τ₂ are parameters calculated in step S61, and e is the bilinear mapping defined in step S11; and if the equation holds true, extracting the country ID and the national alliance chain block identifier BID from the transaction T_{W};
S73. finding the corresponding national alliance chain according to the country ID;
S74. in the national alliance chain, accessing the corresponding block by means of the national alliance chain block identifier BID;
S75. extracting the hash value H₁(C₁) of the ciphertext C₁ of the immunity passport m and the hospital ID from the block;
S76. finding a corresponding hospital server according to the hospital ID, and performing hash-value comparison to obtain the ciphertext C₁=(A,B) of the immunity passport m;
S77. returning the ciphertext C₁ of the immunity passport m to the alliance chain node; and
S78. sending, by the alliance chain node, the ciphertext C₁ of the immunity passport m to the vaccinee.

Further, in step S8, the vaccinee i decrypting the received ciphertext C₁ of the immunity passport to obtain plaintext data m of the immunity passport is as follows:
S81. receiving, by the vaccinee i, the ciphertext C₁=(A,B) of the immunity passport m, wherein A=pkᵢ^{θ} and B=k×m are parameters calculated in step S3, and k=e(g^{θ},H₂(PUIDᵢ)); and
S82. calculating the plaintext data

$$m = \frac{B}{{e ( A , {H}_{2} ( {PUID}_{i} ) )}^{\frac{1}{{sk}_{i}}}}$$

of the immunity passport, where skᵢ is the private key of the vaccinee i, and H₂ is the hash function defined in step S11.

Further, in step S9, the alliance chain node, including the national alliance chain node and the world alliance chain node, performing periodic maintenance by means of a smart contract, checking whether the immunity passport m expires, and updating the expired immunity passport m is specifically as follows:
S91. acquiring, by the alliance chain node, the data of expiry T of the immunity passport m from the transaction;
S92. acquiring, by the alliance chain node, the timestamp t of the transaction;
S93. acquiring, by the alliance chain node, the current time T_{cur}; and
S94. calculating T_{cur}−t; and if T_{cur}−t<T, it indicating that the current immunity passport m has not yet expired; or if T_{cur}−t>T, it indicating that the current immunity passport m has expired, and notifying the relevant hospital to reassess the immune status of the vaccinee i through the national alliance chain node and to update the immunity passport for the vaccinee.

### BRIEF DESCRIPTION OF THE DRAWINGS

FIG. 1 is a schematic flowchart of a blockchain-based supervision method for an electronic immunity passport in an embodiment of the present invention;

FIG. 2 is a schematic illustrative diagram of a blockchain system in the embodiment;

FIG. 3 is a schematic illustrative diagram of an immunity passport in the embodiment;

FIG. 4 is a schematic illustrative structural diagram of a transaction T_{C} in the embodiment; and

FIG. 5 is a schematic illustrative structural diagram of a transaction T_{W} in the embodiment.

### DETAILED DESCRIPTION OF THE INVENTION

A preferred embodiment of the present invention is described below with reference to the accompanying drawings.

### Embodiment

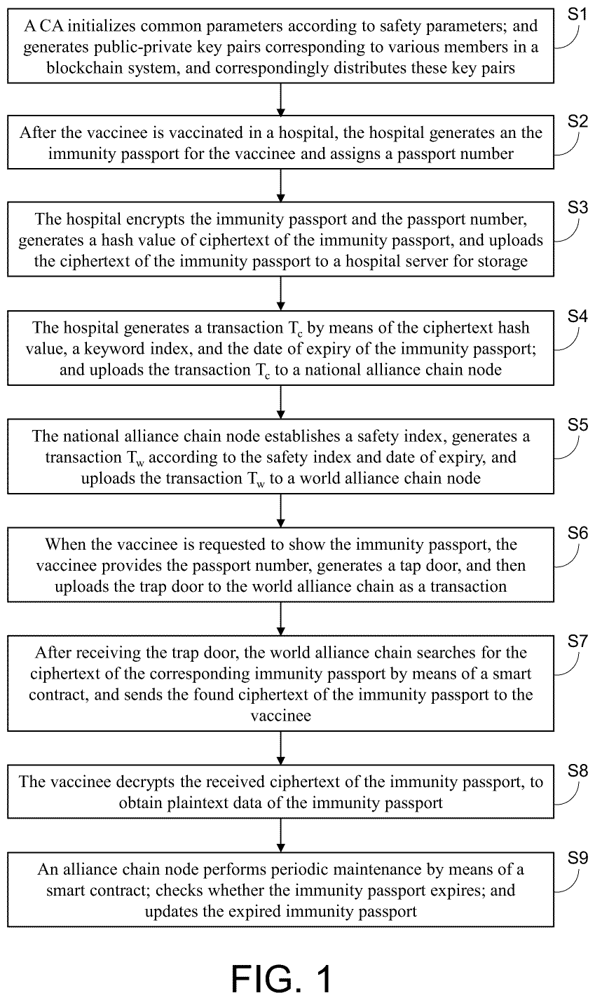

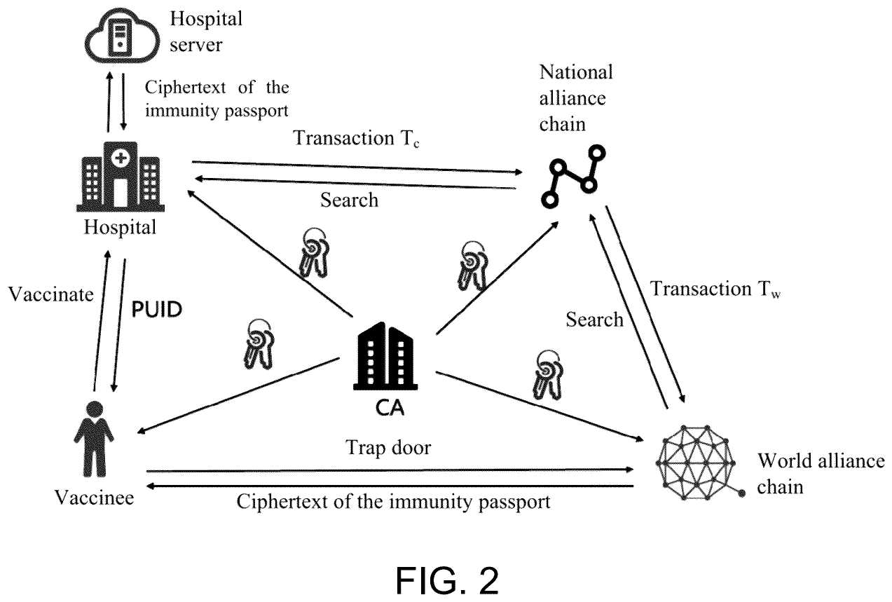

As shown in FIGS. 1 and 2, a blockchain-based supervision method for an electronic immunity passport includes the following steps:
S1. A CA initializes common parameters CP according to safety parameters; and generates public-private key pairs corresponding to various members, including a vaccinee i, a hospital h, a national alliance chain node c and a world alliance chain node w, in a blockchain system, and correspondingly distributes these key pairs. Step S1 is specifically as follows:
S11. The CA selects two multiplicative cyclic groups G₁ and G₂ of which the order is a prime p according to the input safety parameter λ, where a generating element g∈G₁; defines bilinear mapping e: G₁×G₁→G₂; sets three collision-proof hash functions: H₁:{0,1}*→Z*ₚ, H₂: {0,1}*→G₁, H₃: G₂→{0,1}*; and issues the common parameters CP={p, g, e, H₁, H₂, H₃}.
S12. The CA randomly selects xi E Z as a private key skᵢ of the vaccinee i, and calculates the corresponding public key pkᵢ=g^{xi}; randomly selects xₕ∈Z*ₚ as a private key skₕ of the hospital h, and calculates the corresponding key pk_{c}=g^{x}^{_{c}}; randomly selects x_{c} ∈Z*ₚ as a private key sk_{c} of the national alliance chain node c and calculates the corresponding key pk_{c}=g^{x}^{_{c}}; and randomly selects x_{w} ∈Z*ₚ as a private key sk_{w} of the world alliance chain node w and calculates the corresponding key pk_{w}=g^{x}^{_{w}}.

S13. The CA correspondingly assigns, through a secure channel, the public-private key pairs obtained in step S12 to the members, including the vaccinee i, the hospital h, the national alliance chain node c and the world alliance chain node w, in the blockchain system.

S2. After the vaccinee i is vaccinated in the hospital h, the hospital h generates an immunity passport m for the vaccinee i and assigns a passport number PUIDᵢ.

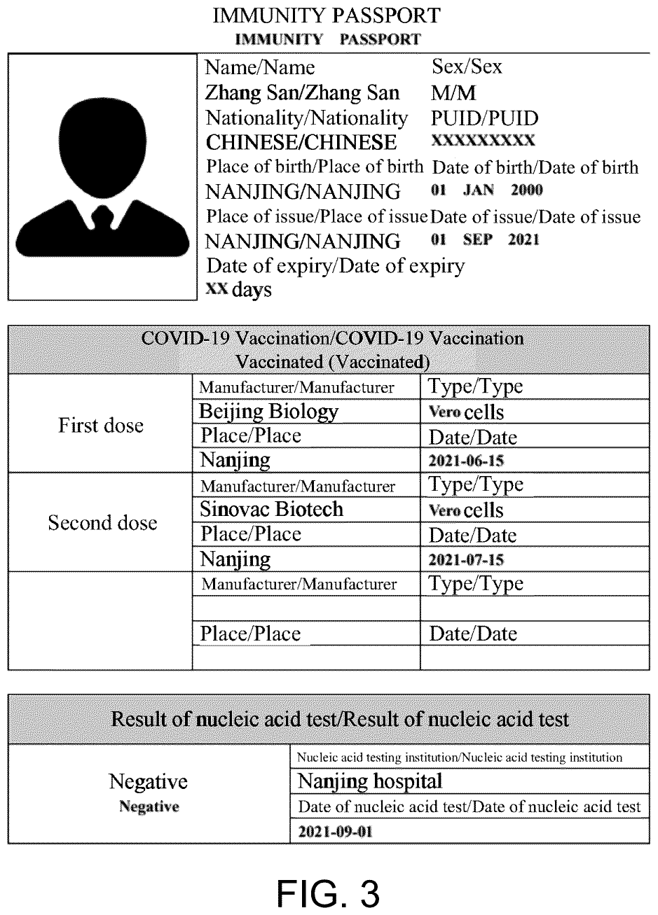

In step S2, as shown in FIG. 3, the immunity passport m includes personal information and vaccination information of a user, where the vaccination information includes vaccination time, vaccination location, and vaccine information; and the vaccine information includes the manufacturer, the type, date of expiry, and the number of vaccination doses.

S3. The hospital h encrypts the immunity passport m and the passport number PUIDᵢ, generates a hash value H₁(C₁) of the ciphertext C₁ of the immunity passport m, and uploads the ciphertext C₁ of the immunity passport m to a hospital server for storage.

In step S3, the hospital h encrypting the immunity passport m and the passport number PUIDᵢ is specifically as follows: The hospital h randomly selects a secrete value θ∈Z*ₚ, where Z*ₚ is the remainder class of the integer module p, that is, Z*ₚ={1,2, . . . , p-1}; calculates A=pkᵢ^{θ}, k=e(g^{θ}, H₂(PUIDᵢ)), B=k×m, and F=H₃(k), where pkᵢ is the public key of the vaccinee i, e is the bilinear mapping defined in step S11, g is the generating element of G₁, G₁ is the multiplicative cyclic group of which the order is a prime p, H₂ and H₃ are two collision-proof hash functions, and m is the plaintext data of the immunity passport; and records C₁=(A,B) as the ciphertext of the immunity passport m and C₂=(A,F) as the ciphertext of the immunity passport number PUIDᵢ.

S4. The hospital h generates a transaction T_{C} by means of the ciphertext hash value H₁(C₁), a keyword index I_{K}, and the date of expiry T of the immunity passport; and uploads the transaction T_{C} to the national alliance chain node. The specific operation is as follows:

S41. The hospital h establishes a keyword index I_{K} [symbol] C₂ [symbol] by means of searchable encryption technology and according to the ciphertext C₂ of the immunity passport number PUIDᵢ.

S42. The hospital h generates a transaction T_{C} by means of the hash value H₁(C₁) of the ciphertext of the immunity passport m, the date of expiry T of the immunity passport, and the keyword index I_{K}. The structure of the transaction T_{C} is shown in FIG. 3.

S43. The hospital h packs the transaction T_{C} and uploads the same to the national alliance chain node. The national alliance chain node runs a PBFT consensus mechanism to authenticate the transaction T_{C}, and generates a new block Block_{C} after successful authentication.

S5. The national alliance chain node establishes a safety index I_{S} by means of searchable encryption technology, generates a transaction T_{W} according to the safety index I_{S} and the date of expiry T, and uploads the transaction T_{W} to the world alliance chain. The specific operation is as follows:

S51. In each national alliance chain, the national alliance chain node extracts a keyword index I_{K}, the data of expiry T, and a block identifier BID of the national alliance chain where the node is located from each transaction in the block Block_{C} generated in step S43.

S52. A safety index I_{S}= [symbol] BID, I_{K} [symbol] is established by means of the keyword index I_{K} and the national alliance chain block identifier BID.

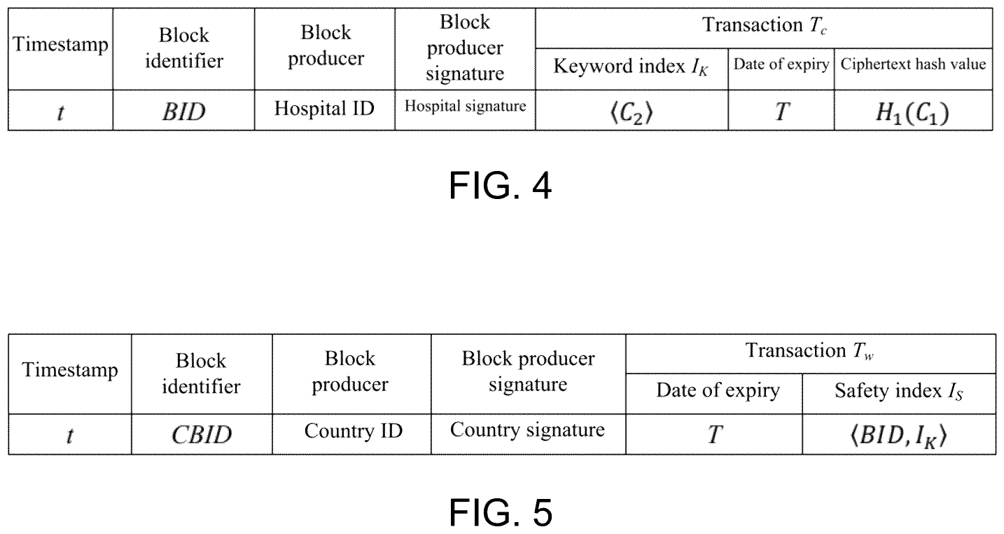

S53. The national alliance chain node generates a transaction T_{W} by means of the safety index I_{S} and the date of expiry T, where the structure of the transaction T_{W} is shown in FIG. 4.

S54. The national alliance chain node packs and uploads the transaction T_{W} to the world alliance chain; and the world alliance chain node runs a PBFT consensus mechanism to authenticate the transaction, and generates a new block Block_{W} after successful authentication.

S6. When the vaccinee i is requested to show the immunity passport m, for example, when the vaccinee i needs to go abroad and is requested to show the immunity passport m, the vaccinee i generates a trapdoor T_{Q} by means of searchable encryption technology and according to the passport number PUIDᵢ, and then uploads the trapdoor T_{Q} to the world alliance chain as a transaction. The specific operation is as follows:

S61. When the vaccinee i is requested to show the immunity passport m, the vaccinee i randomly selects a secrete value d∈Z*ₚ, where Z*ₚ={1,2, . . . , p-1}; and calculates

$${τ}_{1} = {{H}_{2} ( {PUID}_{i} )}^{\frac{1}{{sk}_{i}}} {pk}_{w}^{d}$$

and τ₂=g^{d} by means of his/her own passport number PUIDᵢ, where skᵢ is a private key of the vaccinee i, pk_{w} is a public key of the world alliance chain node w, H₂ is a harsh function defined in step S11, and g is a generating element of G₁.

S62. A trapdoor T_{Q}= [symbol] τ₁, τ₂ [symbol] is generated, where τ₁ and τ₂ are parameters calculated in step S61; and then the trapdoor T_{Q} is uploaded to the world alliance chain as a transaction.

S7. After receiving the trapdoor T_{Q}, the world alliance chain searches for the ciphertext C₁ of the corresponding immunity passport m by means of a smart contract, and sends the found ciphertext C₁ of the immunity passport m to the vaccinee i. The specific operation is as follows:

S71. After receiving the trapdoor T_{Q}= [symbol] τ₁, τ₂ [symbol] , the world alliance chain node calculates

$$\frac{{τ}_{1}}{{{τ}_{2}}^{{sk}_{w}}} ,$$

where sk_{w} is a private key of the world alliance chain node w, and

$${τ}_{1} = {{H}_{2} ( {PUID}_{i} )}^{\frac{1}{{sk}_{i}}} {pk}_{w}^{d}$$

and τ₂=g^{d} are parameters calculated in step S61.

S72. The world alliance chain node extracts the safety index I_{S}= [symbol] BID, I_{K} [symbol] from the transaction T_{W} in the block Block_{W} generated in step S54, to authenticate whether the equation

$$F = {H}_{3} ( e ( A , \frac{{τ}_{1}}{{{τ}_{2}}^{{sk}_{w}}} ) )$$

holds true, where BID is the national alliance chain block identifier, I_{K}= [symbol] C₂ [symbol] is the keyword index, C₂=(A,F) is the ciphertext of the immunity passport number PUIDᵢ, A=pkᵢ^{θ} and F=H₃(k) are parameters calculated in step S3, k=e(g^{θ}, H₂(PUIDᵢ)), sk_{w} is the private key of the world alliance chain node w, τ₁ and τ₂ are parameters calculated in step S61, and e is the bilinear mapping defined in step S11. If the equation holds true, the country ID and the national alliance chain block identifier BID are extracted from the transaction T_{W}.

S73. The corresponding national alliance chain is found according to the country ID.

S74. In the national alliance chain, the corresponding block is accessed by means of the national alliance chain block identifier BID.

S75. The hash value H₁(C₁) of the ciphertext C₁ of the immunity passport m and the hospital ID are extracted from the block.

S76. A corresponding hospital server is found according to the hospital ID, and hash-value comparison is performed to obtain the ciphertext C₁=(A,B) of the immunity passport m.

S77. The ciphertext C₁ of the immunity passport m is returned to the alliance chain node.

S78. The alliance chain node sends the ciphertext C₁ of the immunity passport m to the vaccinee.

S8. The vaccinee i decrypts the received ciphertext C₁ of the immunity passport to obtain plaintext data m of the immunity passport. The specific operation is as follows:

S81. The vaccinee i receives the ciphertext C₁=(A,B) of the immunity passport m, where A=pkᵢ^{θ} and B=k×m are parameters calculated in step S3, and k=e(g^{θ}, H₂(PUIDᵢ)).

S82. The plaintext data

$$m = \frac{B}{{e ( A , {H}_{2} ( {PUID}_{i} ) )}^{\frac{1}{{sk}_{i}}}}$$

of the immunity passport is calculated, where skᵢ is the private key of the vaccinee i, and H₂ is the hash function defined in step S11.

S9. An alliance chain node, including a national alliance chain node and a world alliance chain node, performs periodic maintenance by means of a smart contract; checks whether the immunity passport m expires; and updates the expired immunity passport m. The specific operation is as follows:

S91. The alliance chain node acquires the data of expiry T of the immunity passport m from the transaction.

S92. The alliance chain node acquires the timestamp t of the transaction.

S93. The alliance chain node acquires the current time T_{cur}.

S94. T_{cur}−t is calculated. If T_{cur}−t<T, it indicates that the current immunity passport m has not yet expired; or if T_{cur}−t>T, it indicates that the current immunity passport m has expired, and the relevant hospital is notified to reassess the immune status of the vaccinee i through the national alliance chain node and to update the immunity passport for the vaccinee.

The blockchain-based supervision method for an electronic immunity passport in the embodiment includes the following six entities:

Vaccinee: After being vaccinated, the vaccinee generates his/her immunity passport via a user terminal and acquires his/her own immunity passport number. When the vaccinee plans to go abroad, he/she provides his/her own immunity passport number to an inspector for query, to obtain the ciphertext of the immunity passport and decrypt the ciphertext.

Hospital: After vaccinating the vaccinee against COVID-19, the hospital generates an immunity passport for the vaccinee through a terminal device and encrypts the passport, and uploads the ciphertext data of the immunity passport to the hospital server for storage.

Hospital server: The hospital server stores the ciphertext data of the immunity passport of the vaccinee.

National alliance chain: Each country separately maintains a corresponding national alliance chain which is led by the National Health Commission. The domestic hospitals serve as the national alliance chain nodes and adopt a PBFT consensus mechanism.

World alliance chain: The countries all over the world work together to maintain one world alliance chain which is led by the World Health Organization. The health commission in each country and the international health organizations serve as the world alliance chain nodes and adopt a PBFT consensus mechanism.

Certificate Authority CA: As a trusted third party, the CA initializes common parameters, and generates and distributes keys for each member in the scheme.

The blockchain-based supervision method for an electronic immunity passport stores relevant data, such as indexes and the date of expiry of the immunity passport, by means of blockchain technology, thus ensuring data openness, traceability, and unalterableness, and achieving openness and transparency of the relevant data such as indexes and the date of expiry of the immunity passport. In this way, the immunity passport data can be effectively supervised, and reliable immunity passport data can be provided for the vaccinee, thus achieving secure mutual recognition and communication of the immunity passport between countries, providing convenience for the vaccine, and further avoiding the problems of the forgery of immunity passport data and privacy leakage of personal information data. The method further completes search and regular maintenance for the immunity passport by means of the smart contract of the blockchain.

The blockchain-based supervision method for an electronic immunity passport guarantees data confidentiality by means of searchable encryption technology, implements sharing of the immunity passport data, and further solves the problems of privacy leakage and forgery of personal information data with reference to the cryptographic technique. For the dates of expiry of the vaccine and the immunity passport, the method supervises the transactions on the alliance chain by using smart contract and timestamp technologies, and detects the date of expiry of the immunity passport, so as to know whether and when it is required to reassess the immune status of a passport holder, and to update his/her passport.

### The Present Invention has the Following Advantageous Effects:

1. The blockchain-based supervision method for an electronic immunity passport stores relevant data, such as indexes and the date of expiry of the immunity passport, by means of blockchain technology, thus ensuring data openness, traceability, and immutability. In this way, the immunity passport data can be effectively supervised, and reliable immunity passport data can be provided for the vaccinee, thus achieving secure mutual recognition and communication of the immunity passport between countries, providing convenience for the vaccinee, and further avoiding the problems of forgery of the immunity passport data and privacy leakage of personal information data. The method further completes search and regular maintenance for the immunity passport by means of the smart contract of the blockchain.
2. The blockchain-based supervision method for an electronic immunity passport guarantees data confidentiality by means of searchable encryption technology, implements sharing of the immunity passport data, and further solves the problems of privacy leakage and forgery of personal information data with reference to the cryptographic technique.
3. For the dates of expiry of the vaccine and the immunity passport, the present invention supervises the transactions on the alliance chain by using smart contract and timestamp technologies, and detects the date of expiry of the immunity passport, so as to know whether and when it is required to reassess the immune status of a passport holder and to update his/her passport.
4. In the blockchain-based supervision method for an electronic immunity passport, the immunity passport records personal information and vaccination information of a user, thus facilitating recovery of normal production and life, international contacts and international trade and economic activities.

Although the present invention has been described in detail above with reference to the foregoing embodiment, persons skilled in the art still can modify the technical solutions described in the foregoing embodiment, or make equivalent substitutions for some of the technical features. Any modifications, equivalent replacements, and improvements made within the spirit and principle of the present invention shall fall within the scope of protection of the present invention.

1. A blockchain-based supervision method for an electronic immunity passport, comprising the following steps:
S1. initializing, by a certificate authority (CA), common parameters CP according to safety parameters; and generating public-private key pairs corresponding to various members, including a vaccinee i, a hospital h, a national alliance chain node c and a world alliance chain node w, in a blockchain system, and correspondingly distributing these key pairs;
S2. after the vaccinee i is vaccinated in the hospital h, generating, by the hospital h, an immunity passport m for the vaccinee i and assigning a passport number PUIDᵢ;
S3. encrypting, by the hospital h, the immunity passport m and the passport number PUIDᵢ, generating a hash value H₁(C₁) of ciphertext C₁ of the immunity passport m, and uploading the ciphertext C₁ of the immunity passport m to a hospital server for storage;
S4. generating, by the hospital h, a transaction T_{C} by means of the ciphertext hash value H₁(C₁), a keyword index I_{K}, and the date of expiry T of the immunity passport; and uploading the transaction T_{C} to the national alliance chain node;
S5. establishing, by the national alliance chain node, a safety index I_{S} by means of searchable encryption technology, generating a transaction T_{W} according to the safety index I_{S} and the date of expiry T, and uploading the transaction T_{W} to the world alliance chain;
S6. when the vaccinee i is requested to show the immunity passport m, generating, by the vaccinee i, a trapdoor T_{Q} by means of searchable encryption technology and according to the passport number PUIDᵢ, and then uploading the trapdoor T_{Q} to the world alliance chain as a transaction;
S7. after receiving the trapdoor T_{Q}, searching, by the world alliance chain, for the ciphertext C₁ of the corresponding immunity passport m by means of a smart contract, and sending the found ciphertext C₁ of the immunity passport m to the vaccinee i;
S8. decrypting, by the vaccinee i, the received ciphertext C₁ of the immunity passport to obtain plaintext data m of the immunity passport; and
S9. performing, by an alliance chain node, including a national alliance chain node and a world alliance chain node, periodic maintenance by means of a smart contract; checking whether the immunity passport m expires; and updating the expired immunity passport m.

2. The blockchain-based supervision method for an electronic immunity passport of claim 1, wherein the step S1 is specifically as follows:
S11. selecting, by the CA, two multiplicative cyclic groups G₁ and G₂ of which the order is a prime p according to the input safety parameter λ, a generating element being g E G₁; defining bilinear mapping e: G₁×G₁→G₂; setting three collision-proof hash functions: H₁: {0,1}*→Z*ₚ, H₂: {0,1}*→G₁, H₃: G₂→{0,1}*; and issuing the common parameters CP={p, g, e, H₁, H₂, H₃};
S12. randomly selecting, by the CA, xᵢ∈Z*ₚ as a private key skᵢ of the vaccinee i, and calculating the corresponding public key pk₁=g^{x}ᵢ; randomly selecting xₕ∈Z*ₚ as a private key skₕ of the hospital h, and calculating the corresponding key pk_{c}=g^{x}^{_{c}}; randomly selecting x_{c} ∈Z*ₚ as a private key sk_{c} of the national alliance chain node c and calculating the corresponding key pk_{c}=g^{x}^{_{c}}; and randomly selecting x_{W} ∈Z*ₚ as a private key sk_{w} of the world alliance chain node w and calculating the corresponding key pk_{w}=g^{x}^{_{w}}; and
S13. correspondingly assigning, by the CA through a secure channel, the public-private key pairs obtained in step S12 to the members, including the vaccinee i, the hospital h, the national alliance chain node c and the world alliance chain node w, in the blockchain system.

3. The blockchain-based supervision method for an electronic immunity passport of claim 1, wherein in the step S2, the immunity passport m comprises personal information and vaccination information of a user, the vaccination information comprising vaccination time, vaccination location, and vaccine information.

4. The blockchain-based supervision method for an electronic immunity passport of claim 2, wherein in the step S3, the hospital h encrypting the immunity passport m and the passport number PUIDᵢ is specifically as follows: randomly selecting, by the hospital h, a secrete value θ∈Z*ₚ, wherein Z*ₚ is the remainder class of the integer module p, that is, Z*ₚ={1,2, . . . , p-1}; calculating A=pkᵢ^{θ}, k=e(g^{θ}, H₂(PUIDᵢ)), B=k×m, and F=H₃(k), wherein pkᵢ is a public key of the vaccinee i, e is the bilinear mapping defined in step S11, g is the generating element of G₁, G₁ is the multiplicative cyclic group of which the order is a prime p, H₂ and H₃ are two collision-proof hash functions, and m is the plaintext data of the immunity passport; and recording C₁=(A,B) as the ciphertext of the immunity passport m and C₂=(A,F) as the ciphertext of the immunity passport number PUIDᵢ.

5. The blockchain-based supervision method for an electronic immunity passport of claim 1, wherein in the step S4, the hospital h generating the transaction T_{C} by means of the ciphertext hash value H₁(C₁), the keyword index I_{K}, and the date of expiry T of the immunity passport and uploading the transaction T_{C} to the national alliance chain node is specifically as follows:
S41. establishing, by the hospital h, a keyword index I_{K}= [symbol] C₂ [symbol] by means of searchable encryption technology and according to the ciphertext C₂ of the immunity passport number PUIDᵢ;
S42. generating, by the hospital h, a transaction T_{C} by means of the hash value H₁(C₁) of the ciphertext of the immunity passport m, the date of expiry T of the immunity passport, and the keyword index I_{K}; and
S43. packing, by the hospital h, the transaction T_{C} and uploading the same to the national alliance chain node; and running, by the national alliance chain node, a PBFT consensus mechanism to authenticate the transaction T_{C}, and generating a new block Block_{C} after successful authentication.

6. The blockchain-based supervision method for an electronic immunity passport of claim 1, wherein in the step S5, the national alliance chain node establishing a safety index I_{S} by means of searchable encryption technology, generating a transaction T_{W} according to the safety index I_{S} and the date of expiry T, and uploading the transaction T_{W} to the world alliance chain is specifically as follows:
S51. in each national alliance chain, extracting, by the national alliance chain node, a keyword index I_{K}, the data of expiry T, and a block identifier BID of the national alliance chain where the node is located from each transaction in the block Block_{C} generated in step S43;
S52. establishing a safety index I_{S}= [symbol] BID, I_{K} [symbol] by means of the keyword index I_{K} and the national alliance chain block identifier BID;
S53. generating, by the national alliance chain node, a transaction T_{W} by means of the safety index I_{S} and the date of expiry T; and
S54. packing and uploading, by the national alliance chain node, the transaction T_{W} to the world alliance chain; and running, by the world alliance chain node, a PBFT consensus mechanism to authenticate the transaction, and generating a new block Block_{W} after successful authentication.

7. The blockchain-based supervision method for an electronic immunity passport of claim 2, wherein in the step S6, the vaccinee i generating the trapdoor T_{Q} by means of searchable encryption technology and according to the passport number PUID₁ when the vaccinee i is requested to show the immunity passport m, and then uploading the trapdoor T_{Q} to the world alliance chain as a transaction is specifically as follows:
S61. when the vaccinee i is requested to show the immunity passport m, randomly selecting, by the vaccinee i, a secrete value d∈Z*ₚ, wherein Z*ₚ={1,2, . . . , p-1}; and calculating
$${τ}_{1} = {{H}_{2} ( {PUID}_{i} )}^{\frac{1}{{sk}_{i}}} {pk}_{w}^{d}$$
and τ₂=g^{d} by means of his/her own passport number PUIDᵢ, wherein skᵢ is a private key of the vaccinee i, pk_{w} is a public key of the world alliance chain node w, H₂ is a harsh function defined in step S11, and g is a generating element of G₁; and
S62. generating a trapdoor T_{Q}= [symbol] τ₁, τ₂ [symbol] , wherein τ₁ and τ₂ are parameters calculated in step S61; and then uploading the trapdoor T_{Q} to the world alliance chain as a transaction.

8. The blockchain-based supervision method for an electronic immunity passport of claim 6, wherein in the step S7, the world alliance chain searching for the ciphertext C₁ of the corresponding immunity passport m by means of a smart contract after receiving the trapdoor T_{Q}, and sending the found ciphertext C₁ of the immunity passport m to the vaccinee i is specifically as follows:
S71. after receiving the trapdoor T_{Q}= [symbol] τ₁, τ₂ [symbol] , calculating, by the world alliance chain node,
$$\frac{{τ}_{1}}{{{τ}_{2}}^{{sk}_{w}}} ,$$
wherein sk_{w} is a private key of the world alliance chain node w, and
$${τ}_{1} = {{H}_{2} ( {PUID}_{i} )}^{\frac{1}{{sk}_{i}}} {pk}_{w}^{d}$$
and τ₂=g^{d} are parameters calculated in step S61;
S72. extracting, by the world alliance chain node, the safety index I_{S}= [symbol] BID, I_{K} [symbol] from the transaction T_{W} in the block Block_{W} generated in step S54, to authenticate whether the equation
$$F = {H}_{3} ( e ( A , \frac{{τ}_{1}}{{{τ}_{2}}^{{sk}_{w}}} ) )$$
holds true, wherein BID is the national alliance chain block identifier, I_{K}= [symbol] C₂ [symbol] is the keyword index, C₂=(A,F) is the ciphertext of the immunity passport number PUIDᵢ, A=pkᵢ^{θ} and F=H₃(k) are parameters calculated in step S3, k=e(g^{θ}, H₂(PUIDᵢ)), sk_{w} is the private key of the world alliance chain node w, τ₁ and τ₂ are parameters calculated in step S61, and e is the bilinear mapping defined in step S11; and if the equation holds true, extracting the country ID and the national alliance chain block identifier BID from the transaction T_{w};
S73. finding the corresponding national alliance chain according to the country ID;
S74. in the national alliance chain, accessing the corresponding block by means of the national alliance chain block identifier BID;
S75. extracting the hash value H₁(C₁) of the ciphertext C₁ of the immunity passport m and the hospital ID from the block;
S76. finding a corresponding hospital server according to the hospital ID, and performing hash-value comparison to obtain the ciphertext C₁=(A,B) of the immunity passport m;
S77. returning the ciphertext C₁ of the immunity passport m to the alliance chain node; and
S78. sending, by the alliance chain node, the ciphertext C₁ of the immunity passport m to the vaccinee.

9. The blockchain-based supervision method for an electronic immunity passport of claim 4, wherein in the step S8, the vaccinee i decrypting the received ciphertext C₁ of the immunity passport to obtain plaintext data m of the immunity passport is as follows:
S81. receiving, by the vaccinee i, the ciphertext C₁=(A,B) of the immunity passport m, wherein A=pkᵢ^{θ} and B=k×m are parameters calculated in step S3, and k=e(g^{θ},H₂(PUIDᵢ)); and
S82. calculating the plaintext data
$$m = \frac{B}{{e ( A , {H}_{2} ( {PUID}_{i} ) )}^{\frac{1}{{sk}_{i}}}}$$
of the immunity passport, wherein skᵢ is the private key of the vaccinee i, and H₂ is the hash function defined in step S11.

10. The blockchain-based supervision method for an electronic immunity passport of claim 1, wherein in step S9, the alliance chain node, including the national alliance chain node and the world alliance chain node, performing periodic maintenance by means of a smart contract, checking whether the immunity passport m expires, and updating the expired immunity passport m is specifically as follows:
S91. acquiring, by the alliance chain node, the data of expiry T of the immunity passport m from the transaction;
S92. acquiring, by the alliance chain node, the timestamp t of the transaction;
S93. acquiring, by the alliance chain node, the current time T_{cur}; and
S94. calculating T_{cur}−t; and if T_{cur}−t<T, it indicating that the current immunity passport m has not yet expired; or if T_{cur}−t>T, it indicating that the current immunity passport m has expired, and notifying the relevant hospital to reassess the immune status of the vaccinee i through the national alliance chain node and to update the immunity passport for the vaccinee.

11. The blockchain-based supervision method for an electronic immunity passport of claim 2, wherein in the step S4, the hospital h generating the transaction T_{C} by means of the ciphertext hash value H₁(C₁), the keyword index I_{K}, and the date of expiry T of the immunity passport and uploading the transaction T_{C} to the national alliance chain node is specifically as follows:
S41. establishing, by the hospital h, a keyword index I_{K}= [symbol] C₂ [symbol] by means of searchable encryption technology and according to the ciphertext C₂ of the immunity passport number PUIDᵢ;
S42. generating, by the hospital h, a transaction T_{C} by means of the hash value H₁(C₁) of the ciphertext of the immunity passport m, the date of expiry T of the immunity passport, and the keyword index I_{K}; and
S43. packing, by the hospital h, the transaction T_{C} and uploading the same to the national alliance chain node; and running, by the national alliance chain node, a PBFT consensus mechanism to authenticate the transaction T_{C}, and generating a new block Block_{C} after successful authentication.

12. The blockchain-based supervision method for an electronic immunity passport of claim 3, wherein in the step S4, the hospital h generating the transaction T_{C} by means of the ciphertext hash value H₁(C₁), the keyword index I_{K}, and the date of expiry T of the immunity passport and uploading the transaction T_{C} to the national alliance chain node is specifically as follows:
S41. establishing, by the hospital h, a keyword index I_{K}= [symbol] C₂ [symbol] by means of searchable encryption technology and according to the ciphertext C₂ of the immunity passport number PUIDᵢ;
S42. generating, by the hospital h, a transaction T_{C} by means of the hash value H₁(C₁) of the ciphertext of the immunity passport m, the date of expiry T of the immunity passport, and the keyword index I_{K}; and
S43. packing, by the hospital h, the transaction T_{C} and uploading the same to the national alliance chain node; and running, by the national alliance chain node, a PBFT consensus mechanism to authenticate the transaction T_{C}, and generating a new block Block_{C} after successful authentication.

13. The blockchain-based supervision method for an electronic immunity passport of claim 4, wherein in the step S4, the hospital h generating the transaction T_{C} by means of the ciphertext hash value H₁(C₁), the keyword index I_{K}, and the date of expiry T of the immunity passport and uploading the transaction T_{C} to the national alliance chain node is specifically as follows:
S41. establishing, by the hospital h, a keyword index I_{K}= [symbol] C₂ [symbol] by means of searchable encryption technology and according to the ciphertext C₂ of the immunity passport number PUIDᵢ;
S42. generating, by the hospital h, a transaction T_{C} by means of the hash value H₁(C₁) of the ciphertext of the immunity passport m, the date of expiry T of the immunity passport, and the keyword index I_{K}; and
S43. packing, by the hospital h, the transaction T_{C} and uploading the same to the national alliance chain node; and running, by the national alliance chain node, a PBFT consensus mechanism to authenticate the transaction T_{C}, and generating a new block Block_{C} after successful authentication.

14. The blockchain-based supervision method for an electronic immunity passport of claim 2, wherein in the step S5, the national alliance chain node establishing a safety index I_{S} by means of searchable encryption technology, generating a transaction T_{W} according to the safety index I_{S} and the date of expiry T, and uploading the transaction T_{W} to the world alliance chain is specifically as follows:
S51. in each national alliance chain, extracting, by the national alliance chain node, a keyword index I_{K}, the data of expiry T, and a block identifier BID of the national alliance chain where the node is located from each transaction in the block Block_{C} generated in step S43;
S52. establishing a safety index I_{S}= [symbol] BID, I_{K} [symbol] by means of the keyword index I_{K} and the national alliance chain block identifier BID;
S53. generating, by the national alliance chain node, a transaction T_{W} by means of the safety index I_{S} and the date of expiry T; and
S54. packing and uploading, by the national alliance chain node, the transaction T_{W} to the world alliance chain; and running, by the world alliance chain node, a PBFT consensus mechanism to authenticate the transaction, and generating a new block Block_{W} after successful authentication.

15. The blockchain-based supervision method for an electronic immunity passport of claim 3, wherein in the step S5, the national alliance chain node establishing a safety index I_{S} by means of searchable encryption technology, generating a transaction T_{W} according to the safety index I_{S} and the date of expiry T, and uploading the transaction T_{W} to the world alliance chain is specifically as follows:
S51. in each national alliance chain, extracting, by the national alliance chain node, a keyword index I_{K}, the data of expiry T, and a block identifier BID of the national alliance chain where the node is located from each transaction in the block Block_{C} generated in step S43;
S52. establishing a safety index I_{S}= [symbol] ID, I_{K} [symbol] by means of the keyword index I_{K} and the national alliance chain block identifier BID;
S53. generating, by the national alliance chain node, a transaction T_{W} by means of the safety index I_{S} and the date of expiry T; and
S54. packing and uploading, by the national alliance chain node, the transaction T_{W} to the world alliance chain; and running, by the world alliance chain node, a PBFT consensus mechanism to authenticate the transaction, and generating a new block Block_{W} after successful authentication.

16. The blockchain-based supervision method for an electronic immunity passport of claim 4, wherein in the step S5, the national alliance chain node establishing a safety index I_{S} by means of searchable encryption technology, generating a transaction T_{W} according to the safety index I_{S} and the date of expiry T, and uploading the transaction T_{W} to the world alliance chain is specifically as follows:
S51. in each national alliance chain, extracting, by the national alliance chain node, a keyword index I_{K}, the data of expiry T, and a block identifier BID of the national alliance chain where the node is located from each transaction in the block Block_{C} generated in step S43;
S52. establishing a safety index I_{S}= [symbol] BID, I_{K} [symbol] by means of the keyword index I_{K} and the national alliance chain block identifier BID;
S53. generating, by the national alliance chain node, a transaction T_{W} by means of the safety index I_{S} and the date of expiry T; and
S54. packing and uploading, by the national alliance chain node, the transaction T_{W} to the world alliance chain; and running, by the world alliance chain node, a PBFT consensus mechanism to authenticate the transaction, and generating a new block Block_{W} after successful authentication.

17. The blockchain-based supervision method for an electronic immunity passport of claim 3, wherein in the step S6, the vaccinee i generating the trapdoor T_{Q} by means of searchable encryption technology and according to the passport number PUIDᵢ when the vaccinee i is requested to show the immunity passport m, and then uploading the trapdoor T_{Q} to the world alliance chain as a transaction is specifically as follows:
S61. when the vaccinee i is requested to show the immunity passport m, randomly selecting, by the vaccinee i, a secrete value d∈Z*ₚ, wherein Z*ₚ={1,2, . . . , p-1}; and calculating
$${τ}_{1} = {{H}_{2} ( {PUID}_{i} )}^{\frac{1}{{sk}_{i}}} {pk}_{w}^{d}$$
and τ₂=g^{d} by means of his/her own passport number PUIDᵢ, wherein skᵢ is a private key of the vaccinee i, pk_{w} is a public key of the world alliance chain node w, H₂ is a harsh function defined in step S11, and g is a generating element of G₁; and
S62. generating a trapdoor T_{Q}= [symbol] τ₁, τ₂ [symbol] , wherein τ₁ and τ₂ are parameters calculated in step S61; and then uploading the trapdoor T_{Q} to the world alliance chain as a transaction.

18. The blockchain-based supervision method for an electronic immunity passport of claim 4, wherein in the step S6, the vaccinee i generating the trapdoor T_{Q} by means of searchable encryption technology and according to the passport number PUID₁ when the vaccinee i is requested to show the immunity passport m, and then uploading the trapdoor T_{Q} to the world alliance chain as a transaction is specifically as follows:
S61. when the vaccinee i is requested to show the immunity passport m, randomly selecting, by the vaccinee i, a secrete value d∈Z*ₚ, wherein Z*ₚ={1,2, . . . , p-1}; and calculating
$${τ}_{1} = {{H}_{2} ( {PUID}_{i} )}^{\frac{1}{{sk}_{i}}} {pk}_{w}^{d}$$
and τ₂=g^{d} by means of his/her own passport number PUIDᵢ, wherein skᵢ is a private key of the vaccinee i, pk_{w} is a public key of the world alliance chain node w, H₂ is a harsh function defined in step S11, and g is a generating element of G₁; and
S62. generating a trapdoor T_{Q}= [symbol] τ₁, τ₂ [symbol] , wherein τ₁ and τ₂ are parameters calculated in step S61; and then uploading the trapdoor T_{Q} to the world alliance chain as a transaction.

19. The blockchain-based supervision method for an electronic immunity passport of claim 2, wherein in the step S9, the alliance chain node, including the national alliance chain node and the world alliance chain node, performing periodic maintenance by means of a smart contract, checking whether the immunity passport m expires, and updating the expired immunity passport m is specifically as follows:
S91. acquiring, by the alliance chain node, the data of expiry T of the immunity passport m from the transaction;
S92. acquiring, by the alliance chain node, the timestamp t of the transaction;
S93. acquiring, by the alliance chain node, the current time T_{cur}; and
S94. calculating T_{cur}−t; and if T_{cur}−t<T, it indicating that the current immunity passport m has not yet expired; or if T_{cur}−t>T, it indicating that the current immunity passport m has expired, and notifying the relevant hospital to reassess the immune status of the vaccinee i through the national alliance chain node and to update the immunity passport for the vaccinee.

20. The blockchain-based supervision method for an electronic immunity passport of claim 3, wherein in the step S9, the alliance chain node, including the national alliance chain node and the world alliance chain node, performing periodic maintenance by means of a smart contract, checking whether the immunity passport m expires, and updating the expired immunity passport m is specifically as follows:
S91. acquiring, by the alliance chain node, the data of expiry T of the immunity passport m from the transaction;
S92. acquiring, by the alliance chain node, the timestamp t of the transaction;
S93. acquiring, by the alliance chain node, the current time T_{cur}; and
S94. calculating T_{cur}−t; and if T_{cur}−t<T, it indicating that the current immunity passport m has not yet expired; or if T_{cur}−t>T, it indicating that the current immunity passport m has expired, and notifying the relevant hospital to reassess the immune status of the vaccinee i through the national alliance chain node and to update the immunity passport for the vaccinee.
